(19) United States
(12) Patent Application Publication
(10) Pub. No.: US 2014/0080211 A1
(43) Pub. Date: Mar. 20, 2014

(54) MULTIWELL PLATE FOR REMOVING LIQUID AND CELL CULTURE METHOD USING THE SAME

(71) Applicant: Samsung Electronics Co., Ltd., Suwon-si (KR)

(72) Inventors: Moon-sook LEE, Seoul (KR); Eun KO, Anyang-si (KR); Min-sang KIM, Anseong-si (KR)

(21) Appl. No.: 14/029,323

(22) Filed: Sep. 17, 2013

(30) Foreign Application Priority Data
Sep. 17, 2012 (KR) 10-2012-0102992

(51) Int. Cl. C12M 1/00; C12N 5/07

(52) U.S. Cl. CPC C12M 21/00; C12N 5/06
USPC 435/383; 435/305.2

(57) ABSTRACT

Provided is a multiwell plate that includes first wells and second wells separated by a sidewall made of a porous material and a method of using the same.

### CROSS-REFERENCE TO RELATED APPLICATIONS

This application claims the benefit of Korean Patent Application No. 10-2012-0102992, filed on Sep. 17, 2012, in the Korean Intellectual Property Office, the disclosures of which are incorporated herein in their entirety by reference.

### BACKGROUND

1. Field

The present disclosure relates to a multiwell plate easy to remove liquid and a cell culture method using the same.

2. Description of the Related Art

Experimental plates including multiwells or reaction chambers are used for various purposes and analyses, including cell culture. When cells are cultured in wells of multiwell plates, media used in the wells may need to be exchanged regularly. A manual or automated apparatus may be used to remove the used media from the wells and supply new media. In order to exchange media when suspension cells grow in a suspended state in a medium, the culture may be centrifuged to precipitate cells such that only a supernatant may be removed. However, this process is inefficient when it must be performed for each well of multiwell plates. Therefore, there remains a demand for multiwell plates enabling liquid to be more efficiently removed.

### SUMMARY

Provided is a multiwell plate for easily removing liquid from a sample or culture contained in well. In one aspect, the multiwell plate comprises a plurality of first wells and a plurality of second wells, each first well being separated from a second well by a common first sidewall, wherein at least a portion of the first sidewall comprises one or more pores impermeable to cells and permeable to a liquid component, thereby enabling a liquid component to be transferred from the first wells to the second wells.

In another aspect, the multiwell plate comprises a plurality of first wells and a plurality of second wells, each first well being separated from a second well by a common first sidewall, wherein each first well has a bottom, and at least a portion of the bottom of the first well comprises one or more pores impermeable to cells and permeable to a liquid component, thereby enabling a liquid component to be transferred out of the first well.

Also provided is a cell culture method using the multiwell plate. According to one aspect, method comprises culturing cells in a medium in a first well of the multiwall plate, and transferring the medium in the culture from the first well to the second well through the one or more pores in the first sidewall. According to another aspect, the method comprises culturing cells in a medium in a first well of the multiwall plate, and transferring the medium in the culture from the first well through one or more pores in the bottom surface of the first well.

Additional aspects of the invention will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1 is a schematic view illustrating a multiwell plate;

FIG. 2 is a schematic view illustrating a multiwell plate having a first sidewall door;

FIGS. 3 and 4 are side views in A and B directions of FIG. 2, respectively;

FIG. 5 is a schematic view illustrating a multiwell plate including first chambers 28, 28′ under second wells 22, 22′;

FIG. 6 is a schematic view illustrating a state where the bottom of the second well 30 in the multiwell plate of FIG. 5 is sealed;

FIG. 7 is a schematic view illustrating a state where the bottom 30 of the second well 22 in the multiwell plate of FIG. 6 is opened;

FIG. 8 is a schematic view illustrating a state where the bottom of the second well 30 in the multiwell plate of FIG. 5 is sealed;

FIG. 9 is a schematic view illustrating a state where the bottom 30 of the second well 22 in the multiwell plate of FIG. 8 is opened;

FIG. 10 is a schematic view illustrating a multiwell plate having electrodes at each of the second wells;

FIG. 11 is a schematic view illustrating a multiwell plate having one or more pores in an area of the bottom of each of the first wells;

FIG. 12 is a schematic view illustrating a multiwell plate in which a bottom of a second well is positioned at a lower level than a bottom of a first well; and

FIGS. 13 to 15 are schematic views illustrating a method of manufacturing a multiwell plate.

### DETAILED DESCRIPTION

According to an aspect of the present invention, a multiwell plate is provided. As a general matter, the multiwall plate can be described as having a top (although the wells may be open-top wells), a bottom, and a thickness between the top and the bottom. The multiwall plate has a plurality of first wells and second wells extending through (in the direction of) the thickness of the plate. Each well may be generally defined by sidewalls (e.g., 2 or more, 3 or more, or 4 or more sidewalls) and a bottom. According to one aspect, each of the first wells of the multiwell plate are separated from a second well by a common (shared) first sidewall, and at least a portion of the first sidewall having one or more pores impermeable to cells and permeable to a liquid component, thereby enabling the liquid component to be transferred from the first wells to the second wells. The one or more pores can be provided by a porous material that serves as a portion of the first sidewall.

The multiwell plate may include an arrangement of the first wells and the second wells. The arrangement of the first wells and the second wells may be a uniform arrangement, for example, a parallel or non-parallel arrangement. For example, the multiwell plate may be a 6 well, a 24 well, a 48 well, or a 96 well plate well known in the art. The size of the first wells and the second wells determining the amount of liquid or material for storage therein may be appropriately selected based on the desired end use. A portion of or all of the multiwell plate may be made of a moldable material, for example, plastic. Examples of moldable materials include those selected from the group consisting of polyethylene, polypropylene, polystyrene, and any combinations thereof. The first wells may be designed to be able to observe cell culture or cells being cultured therein. For example, at least the first wells of the multiwell plate may be transparent and be capable to transmit light or a magnetic field. The second wells may or may not be made of the same material or have the same size as the first wells. The first wells and/or second wells may be made of materials suitable for cell culture. The cells may be animal cells. The shape of the first wells and/or second wells on a plan view may be circular or polygonal. For example, the shape may be rectangular or pentagonal.

In the multiwell plate, a portion of or all of the first sidewall may be made of a porous material having one or more pores impermeable to cells and permeable to a liquid component, which enables the liquid component to be transferred from the first wells to the second wells while preventing passage of cells. The cells may be prokaryotic cells or eukaryotic cells. For example, the cells may be animal cells. The animal cells may be suspension cells or adherent cells. The animal cells may be human, rat, bovine, hog, horse, rabbit, or goat cells. The first sidewall may have the form of a porous membrane. For example, the first sidewall may be a flat sheet having a thickness ranging from about 5 μm to about 100 μm, about 5 μm to about 80 μm, about 5 μm to about 60 μm, about 5 μm to about 40 μm, about 5 μm to about 20 μm or about 5 μm to about 10 μm. The average diameter of the pores in the porous material of the first sidewall may be in a range of about 1 μm to about 25 μm, about 2 μm to about 20 μm, about 3 μm to about 15 μm, about 4 μm to about 10 μm, about 5 μm to about 8 μm, about 5 μm to about 25 μm, about 2 μm to about 20 μm, about 3 μm to about 15 μm, about 4 μm to about 10 μm, or about 4 μm to about 10 μm. The porous material may be a biocompatible medical mesh material, for example, polyesters such as polypropylene (PP) and polyethylene terephthalate (PET), polyamides such as nylon, polytetrafluoroethylene (PTFE), polyetheretherketone (PEEK), polyetherketoneketone (PEKK), silk, metallic wires (for example, stainless steel, nitinol, and Pt—Ir) or any combinations.

The multiwell plate may include a first sidewall door closingly and sealably disposed in the second well by which liquid movement between the first wells and the second wells through the pores in the first sidewell can be controlled and adjusted. By closingly and sealably disposed, it is meant that the sidewall door can be opened and closed, and, when closed, seals or blocks the pores or the area containing the pores or porous material such that liquid cannot pass between the first and second wells. Thus, when the first sidewall door is closed relative to the first sidewall, liquid cannot move through the pores from the first to the second wells, and if the first sidewall door is open, it is spaced apart from the first sidewall such that liquid may not move to the second wells. When the first sidewall door is closed, the first well is fluidically disconnected from the second well, but if the first sidewall door is opened, the first and second wells are fluidically connected via the pores. The first sidewall door may be made of a material having a membrane shape. For example, the first sidewall door may be a membrane with a size corresponding to that of the first sidewall. Also, if the first sidewall door is closed, the first sidewall door maybe sealed to second wells such that liquid may not move to the rest portions of the second wells.

In the multiwell plate, the first sidewall door may be disposed to be rotatable from the bottom of the second wells in the top direction with a top portion thereof serving as a rotation axis. By way of further illustration, the first sidewall door may have a bottom edge positioned nearest the bottom of the second well when closed and a top edge opposite the bottom edge and closest to a top portion of the second well. The first sidewall door may be disposed with a rotational axis along the top edge, such that the bottom edge of the first sidewall door rotates or swings away from the first sidewall and into the space of the second well when opened. The first sidewall door may be opened or closed mechanically or electrically. For example, if the first sidewall door is made of an electroactive material that bends when a voltage is applied thereto, and each of the first sidewalls may be disposed between a pair of electrodes in order to individually receive the voltage. Upon application of a voltage, the sidewall door may open or close. The electroactive material may be an electrically active hydrogel. For example, the electroactive material may be a hydrogel including a polymer selected from the group consisting of acryl acid, methacryl acid and any combinations. The electroactive material may be a hydrogel made of a conductive polymer.

In the multiwell plate, the second wells may have a second sidewall that is not in contact with a first well, and that may be opened or closed. The shape of the second wells on a plan view may be circular or polygonal. For example, the shape may be rectangular or pentagonal. If the case is rectangular, the second sidewall may correspond to a side of the rectangular shape. If the second sidewall is opened, the liquid of the second wells may be transferred to other neighboring second wells or outside of the multiwell plate. The movement may be produced by a suctioning apparatus. Therefore, a suctioning pump may be connected to the second wells.

In the multiwell plate, the second sidewall door may be disposed to be rotatable from the bottom of second wells in the top direction with the top portion thereof serving as a rotation shaft. By way of further illustration, the second sidewall door may have a bottom edge nearest the bottom of the second well when closed and a top edge opposite the bottom edge, and the second sidewall door is disposed with a rotational axis along the top edge, such that the bottom edge of the first sidewall door swings away from the first sidewall and into or away from the second well when opened. The second sidewall door may be opened and closed mechanically or electrically.

If the second sidewall is made of an electroactive material that bends when a voltage is applied thereto, the second sidewall may be opened or closed electrically. Each of the second sidewalls may be disposed between a pair of electrodes in order to individually receive a voltage. Each second sidewall may be opened and closed individually. The electroactive material may be an electrically active hydrogel. For example, the electroactive material may be a hydrogel including a polymer selected from the group consisting of acryl acid, methacryl acid and any combinations. The electroactive material may be a hydrogel made of a conductive polymer.

A bottom of at least one of the second wells may be reversibly sealably opened or closed. The bottom may be opened and closed mechanically or electrically. The bottom may be made of an electroactive material that bends when a voltage is applied thereto to be opened or closed electrically. The electroactive material may be a hydrogel including a polymer selected from the group consisting of acryl acid, methacryl acid and any combinations. The electroactive material may be a hydrogel made of a conductive polymer.

The bottoms may be respectively disposed between a pair of electrodes to individually receive a voltage. The pair of electrodes disposed for each of the bottoms may be connected to a power source and/or controller. Each of the bottoms may be opened and closed individually. If each of the bottoms is opened, a liquid within the second wells may move downwards or may be transferred downwards via suction.

The multiwell plate may have a first chamber located under the second well, the first chamber including a top defined by the bottom of the second well, a bottom, and sidewalls. The top of the second well may be sealed, the bottom of the second well may be disposed between a pair of electrodes, one of the pair of electrodes may be located in an upper or top region of the second well, and the other of the pair of electrodes may be located on the bottom of the first chamber. One or more selected from the group consisting of the bottom of the first chamber, the sidewalls, and any combinations may be opened or closed.

According to another aspect of the invention, a cell culture system includes the above multiwell plate, a cell culture measuring apparatus disposed to observe or analyze cell culture in the first wells of the multiwell plate, and a controller connected to open or close at least one of the first sidewall doors of the second well, the sidewalls, and the bottom of the second well (e.g., by applying a voltage to the electrodes positioned to open or close the doors or bottom).

The measuring apparatus may be selected from the group consisting of a microscope, an optic measuring apparatus, an electrical measuring apparatus, a magnetic measuring apparatus and any combinations.

The controller may control an electrical signal, for example, selected from the group consisting of current, voltage, resistance and any combinations.

According to another aspect of the invention, a cell culture method includes: culturing cells in a medium in a first well of a multiwell plate as described herein; transferring the medium in a culture from first wells to the second wells through the pores of the first sidewall; and removing the medium transferred to the second well.

The multiwell plate may be configured as described above. The cell culture may be performed by methods known in the art. The culture may be performed with or without stirring. The cells may be animal cells. The animal cells may be suspension cells or adherent cells. Therefore, the culture may be performed by adhering adherent cells on the first wells, or by suspending suspension cells in a medium of first wells.

The method may include transferring the medium in a culture from first wells to second wells through the pores of the first sidewall. The transferring may be controlled by opening or closing the first sidewall door. The controlling may be performed by controlling the level or degree of opening the first sidewall door based on cell culture conditions in the first well. The cell culture condition may be a factor selected from the group consisting of cell, cell growth rate, pH, cell concentration and any combinations. Therefore, the transferring may further include opening or closing the first sidewall door based on the cell culture condition by using a controller connected to the first sidewall door. Also, the controlling may be transferring the medium to the second well by opening the first sidewall door as much as the quantity of the medium introduced to the first well. Therefore, the method may further include supplementing the medium to the first well. Also, the method may further include supplementing the medium to the first well in a quantity equal to the quantity of the medium removed from the second well.

Also, the method may further include disposing a cell culture observing apparatus to observe cells in the first wells, for example, an optic observing apparatus. The apparatus may be selected from the group consisting of a microscope, an optic measuring apparatus, an electrical measuring apparatus, a magnetic measuring apparatus and any combinations.

The method also may include removing the medium transferred to the second wells. The medium of each of the second wells may be suctioned and removed individually. The medium of each of the second wells may be removed by moving downward due to the gravity when the bottom of the second well is opened. The opening may be performed electrically. The medium of each of the second wells may be removed by being transferred to neighboring second wells or to the outside of the multiwell plate when the sidewall of the second wall is opened.

According to another aspect of the present invention, the multiwell plate includes a plurality of first wells and a plurality of second wells, wherein each first well is separated from a second well by a common first sidewall, and at least a portion of the bottom of the first well has one or more pores impermeable to cells and permeable to a liquid component, thereby enabling the liquid component to be transferred from the first wells to another space outside of the first well. As with the other aspects of the invention, the multiwall plate can be described as having a top (although the wells may be open-top wells), a bottom, and a thickness between the top and the bottom, and plurality of first wells and second wells extend through (in the direction of) the thickness of the plate. Each well may be generally defined by sidewalls (e.g., 2 or more, 3 or more, or 4 or more sidewalls) and a bottom.

A portion of or all of the bottom of the first well may be made of a porous material that provides the one or more pores impermeable to cells and permeable to a liquid component, thereby enabling the liquid component to be transferred from the first wells to the outside of the first wells through the porous material. The cells may be prokaryotic cells or eukaryotic cells. For example, the cells may be animal cells. The animal cells may be suspension cells or adherent cells. The animal cells may be human, rat, bovine, hog, horse, rabbit or goat cells. The bottom of the first well may, in whole or in part, have the form of a porous membrane. For example, the bottom of the first well or portion thereof may be a flat sheet form having a thickness ranging from about 5 μm to about 100 μm, about 5 μm to about 80 μm, about 5 μm to about 60 μm, about 5 μm to about 40 μm, about 5 μm to about 20 μm or about 5 μm to about 10 μm. The average diameter of pores in the porous material may be in a range of about 1 μm to about 25 μm, about 2 μm to about 20 μm, about 3 μm to about 15 μm, about 4 μm to about 10 μm, about 5 μm to about 8 μm, about 5 μm to about 25 μm, about 2 μm to about 20 μm, about 3 μm to about 15 μm, about 4 μm to about 10 μm, or about 4 μm to about 10 μm. The porous material may be a biocompatible medical mesh material, for example, polyesters such as polypropylene (PP) and polyethylene terephthalate (PET), polyamides such as nylon, polytetrafluoroethylene (PTFE), polyetheretherketone (PEEK), polyetherketoneketone (PEKK), silk, metallic wires (for example, stainless steel, nitinol, and Pt—Ir) or any combinations.

The portion of the first well may be provided with one or more pores by by forming a pore or other opening of any suitable shape in the bottom of the first well and filling or covering the pore with a porous material.

The multiwell plate may include a bottom opening/closing door which is closingly and sealably disposed against the bottom of the first well or portion thereof that includes the one or more pores so that, when closed, the liquid component in the first wells may not pass to the second well through the pores, or, when open, may be spaced apart from the bottom of the first well so that liquid component may move to the second wells through pores. The bottom opening/closing door may be a solid support made of a compressible or elastic material. For example, the bottom opening/closing door may be a solid support that may cover the porous material and be sealable such that liquid may not move downward from the first well through the pores.

The bottom opening/closing door may be disposed rotatable downward from the bottom with one end serving as a rotation shaft. In other words, the bottom door comprising one or more edges along the periphery of the door is disposed with a rotational axis along one edge, such that the door swings away from the bottom of the first well in a downward direction away from the first well.

The bottom opening/closing door may be opened or closed mechanically, electrically, or electromechanically. The bottom opening/closing doors may be connected to a power source to individually control closing or opening. An electrical switch may be connected to each of the bottom opening/closing door in order to individually control closing or opening. The electrical switch may be a transistor.

The bottom opening/closing door may be made of an electroactive material that bends when a voltage is applied thereto to be opened or closed electrically. The electroactive material may be a hydrogel selected from the group consisting of acryl acid, methacryl acid and any combinations. The electroactive material may be a hydrogel made of a conductive polymer.

The multiwell plate may have a space (e.g., an enclosed space) under the bottom opening/closing door such that the bottom opening/closing door is rotatable and swings open into the space. The space may be in fluid-communication with the second well, or may be in fluid-communication with a space or chamber under the second well. At least a portion of the multiwall plate under the bottom (opposite the interior of the first well relative to the bottom of the first well, and separated from the first well by the bottom of the first well) of the first well may be supported by a supporter. The supporter may be a solid support that mechanically supports the first well. The solid support may be optically transparent. The solid support may be an insulator.

The multiwell plate may include two or more electrodes, and at least two electrodes may be disposed such that the bottom opening/closing door is located therebetween. Two electrodes may be disposed on the first sidewall in the second well. Alternatively, at least one electrode may be disposed on the first sidewall in the second well, and at least one electrode may be disposed in the bottom space of the second well below the first sidewall door.

The bottom opening/closing door may be extended in the second well direction to exceed the extension line of the first sidewall. In other words, when in the closed position, the bottom door is positioned adjacent a bottom surface of the bottom of the first well and extends parallel to the bottom surface in the direction of the second well beyond the extension line of the first sidewall (beyond the point at which the first sidewall joins the bottom of the first well). In this position, the bottom door may extend into the second well, or into a chamber below the second well.

The bottom of the first well may be positioned at the same level as the bottom of the second well. Also, the bottom of the second well may be positioned at a lower level than the bottom of the first well such that the first well is in fluid-communication with the space provided under the bottom opening/closing door.

According to another aspect of the present invention, a cell culture method includes: culturing cells in a medium in first wells of a multiwell plate described herein; transferring the medium of a culture from the first wells of the plate via pores in first sidewalls; and, optionally, removing the medium transferred to the outside.

The method includes culturing cells in a medium in the first wells of the multiwell plate. The cell culture may be performed by methods known in the art. The culture may be performed with or without stirring. The cells may be animal cells. The animal cells may be suspension cells or adherent cells. Therefore, the culture may be performed by adhering adherent cells on the first well, or by suspending suspension cells in a medium of the first well.

The method may include transferring the medium in a culture from the first wells to second wells through the pores of porous material. The transferring may be controlled by sealing (closing) or spacing (opening) the bottom opening/closing door. The closing or opening may be performed mechanically, electrically energy, or electromechanically. The controlling may be performed by controlling the level of opening the bottom opening/closing door based on cell culture conditions in the first well. The cell culture condition may be a factor selected from the group consisting of cell, cell growth rate, pH, cell concentration and any combinations. Therefore, the transferring may further include opening or closing the bottom opening/closing door based on the cell culture condition by using a controller connected to the bottom opening/closing door. Also, the controlling may be transferring the medium to the outside of the first well by opening the bottom opening/closing door as much as the quantity of the medium introduced to the first well. Therefore, the method may further include supplementing a medium to the first well. Also, the method may further include supplementing a medium to the first well in a quantity equal to the quantity of the medium removed from the second well.

Also, the method may further include disposing a cell culture observing apparatus to observe or analyze cells in the first wells, for example, an optic observing apparatus. The apparatus may be selected from the group consisting of a microscope, an optic measuring apparatus, an electrical measuring apparatus, a magnetic measuring apparatus and any combinations.

The method also includes removing the medium transferred to the outside. The removing may be individually suctioning and removing the liquid component from the space where the liquid component is transferred from the first well. The medium of each of the first wells may be removed by moving downward by gravity when the bottom of the second well is opened. The opening may be performed electrically. A means removing the liquid component may be disposed in the space. The means may be a pump, a suctioning apparatus, or any combination.

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

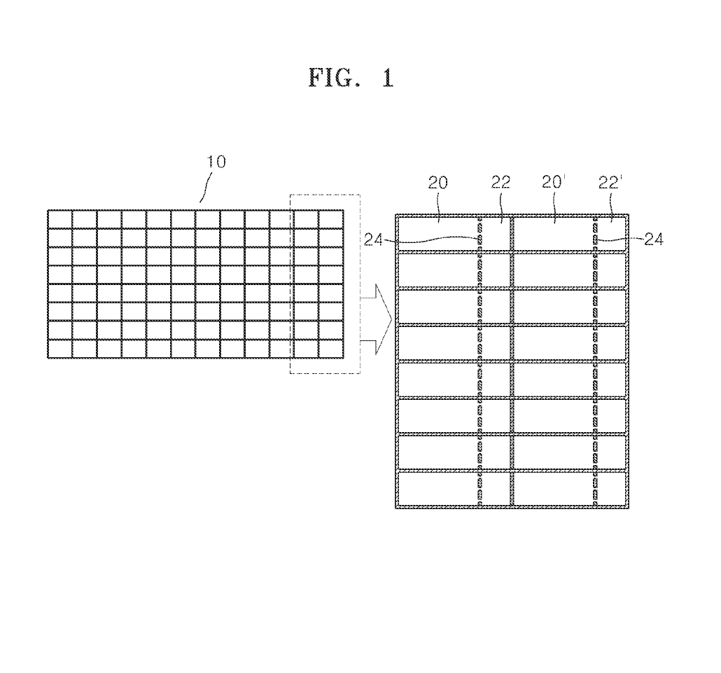

FIG. 1 is a schematic view illustrating a multiwell plate according to an exemplary example. A multiwell plate 10 includes a plate having a top, a bottom, a thickness between the top and the bottom, and a plurality of first wells 20, 20′ and second wells 22, 22′ extending in the direction of the thickness of the plate, first wells 20, 20′ and at least one second wells 22, 22′ being separated by a common first sidewall 24, and the at least a portion of the first sidewall 24 being made of a porous material having one or more pores impermeable to cells and permeable to a liquid component, thereby enabling the liquid component to be transferred from the first wells 20, 20′ to the second wells 22, 22′.

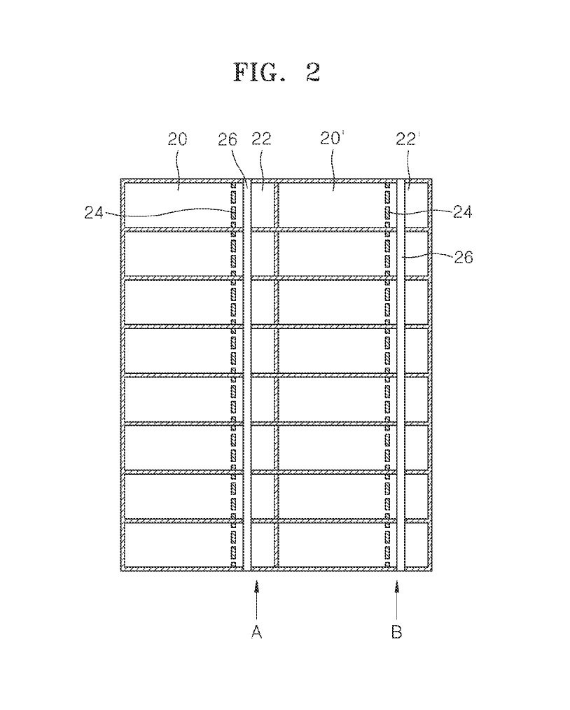

FIG. 2 is a schematic view illustrating a multiwell plate having a first sidewall door according to an exemplary example. In FIG. 2, a first sidewall door 26 is disposed in the second wells 22 at the left spaced apart from the first sidewall 24, but both ends of the first sidewall door 26 are sealed to the sidewalls of the second wells 22, so that fluid may not move to other regions of the second wells 22. The first sidewall door 26 may be closingly and sealably disposed so that liquid component in the first wells 20 may not move to the second wells through pores reversibly, or may be spaced apart so that liquid component may move to the second wells 22 through pores. The first sidewall door 26 may be rotatable from the bottom of the second wells 22 towards the top with the top portion thereof serving as a rotation shaft. The second wells 22′ at the right in FIG. 2 are in a state where the first sidewall door 26 spaced apart from the first sidewall 24 is opened by rotating from the bottom of the second wells 22′ towards the top with the top portion thereof serving as a rotation axis. Consequently, fluid in the first wells 20′ may be transferred to the second wells 22′.

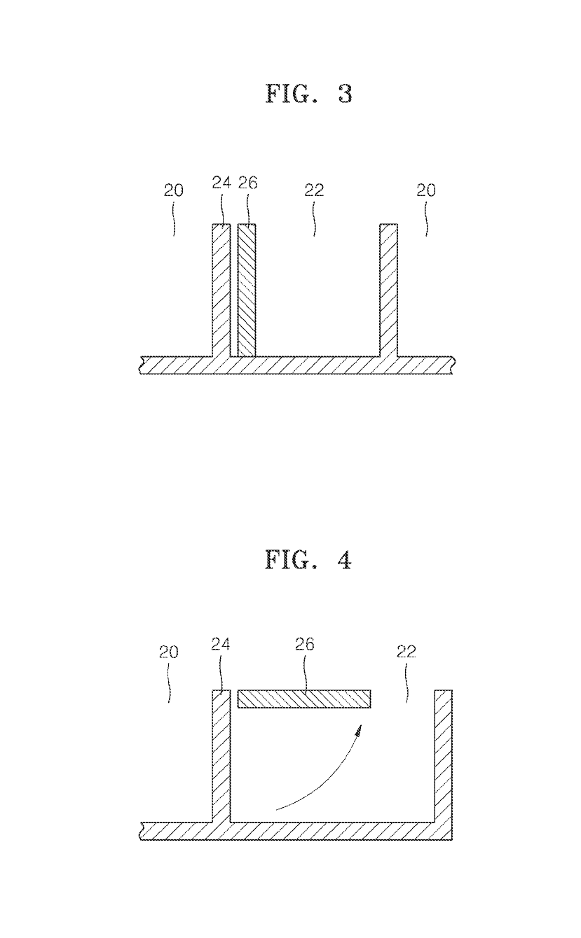

FIGS. 3 and 4 are side views from A and B directions of FIG. 2, respectively. According to FIG. 3, the first sidewall door 26 is spaced apart from the first sidewall 24 and is sealed to the bottom of the second well 22 and both sidewalls of the second well 22, so that fluid may not move from the first well 20 to the second well 22 through the pores of the first sidewall 24.

According to FIG. 4, the first sidewall door 26 is in an opened state by rotating from the bottom of the second well 22 towards the top (i.e., in the arrow direction) with the top portion thereof serving as a rotation axis. Consequently, liquid in the first well 20 may be transferred to the second well 22 through the pores of the first sidewall door 24.

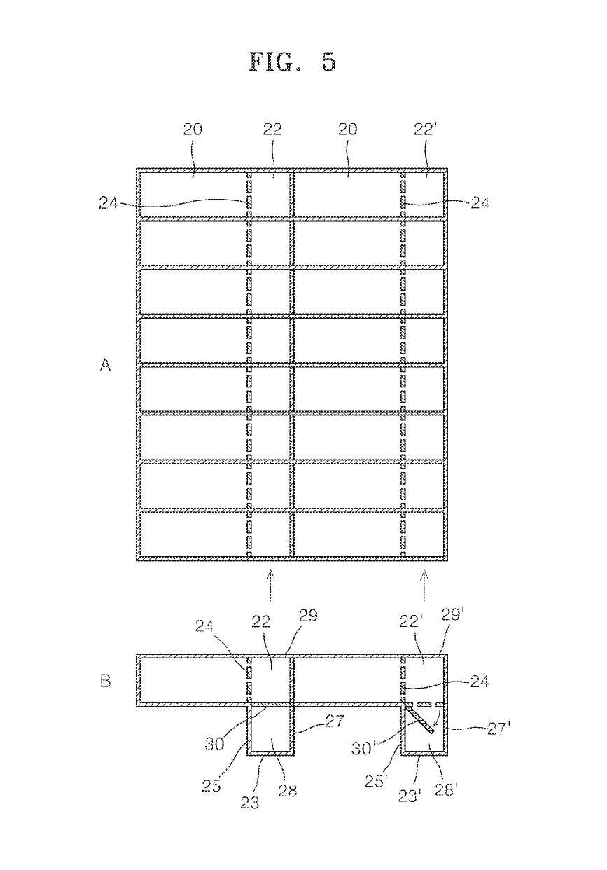

FIG. 5 is a schematic view illustrating a multiwell plate including first chambers 28, 28′ under second well 22, 22′ according to an exemplary example, in which “A” is a plane view and “B’ is a side view viewed in the arrow direction.

According to FIG. 5, the multiwell plate may have the first chambers 28, 28′ located under the second wells 22, 22′, and the first chambers 28, 28′ may include top faces 30, 30′ defined by the bottoms of the second wells 22, 22′, and bottoms (23, 23′) and sidewalls (25, 27, 25′, 27′). Tops (29, 29′) of the second wells 22, 22′ may be closed or opened. The bottom 30, 30′ of each of the second wells 22, 22′ may be disposed between a pair of electrodes, one being located on the top of the second wells 22, 22′ and the other being located on the bottoms of the first chambers 28, 28′. One or more parts selected from the group consisting of a bottom, sidewalls and combinations thereof may be openable and closable.

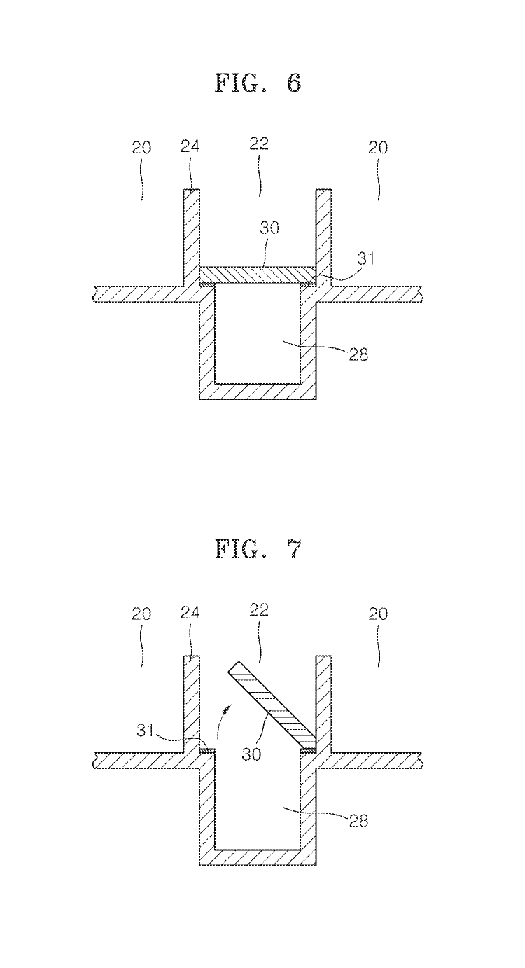

FIG. 6 is a schematic view illustrating a state where the bottom of the second well 30 in the multiwell plate of FIG. 5 is closed. According to FIG. 6, the bottom 30 of the second well 22 is supported by a protruding periphery 31 while sealing the first chamber 28 and at the same time being sealed with respect to the sidewalls of the second well 22 not to allow liquid from moving to the first chamber 28. The protruding periphery 31 is optional, and the present invention may be configured without the protruding periphery 31. The bottom 30 may be opened or closed mechanically or electrically. The bottom 30 may be made of an electroactive material that bends when a voltage is applied thereto such that it may be opened or closed electrically. The electroactive material may be an electroactive hydrogel. For example, the electroactive material may be a hydrogel including a polymer selected from the group consisting of acryl acid, methacryl acid, and combinations thereof. The electroactive material may be a hydrogel made of a conductive polymer.

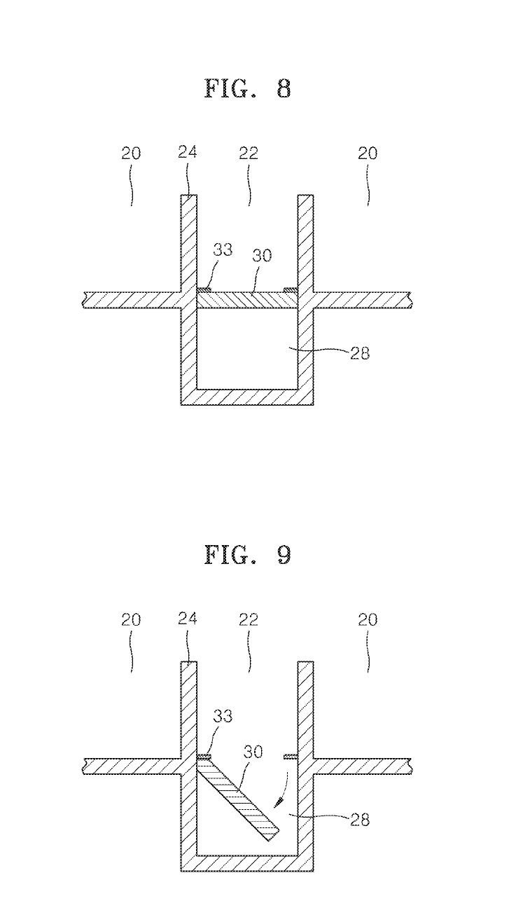

FIG. 7 is a schematic view illustrating a state where the bottom 30 of the second well 22 in the multiwell plate of FIG. 6 is opened. According to FIG. 7, the second well 22 is opened by the bottom 30 thereof rotating upward with the right end of the bottom 30 serving a rotation axis, so that liquid in the first well 20 may be transferred to the first chamber 28 through the pores of the first sidewall 24.

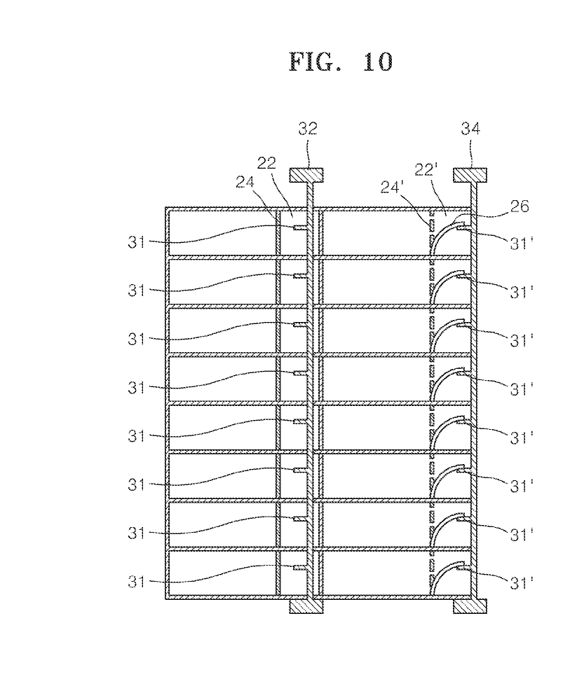

FIG. 8 is a schematic view illustrating a state where the bottom of the second well 30 in the multiwell plate of FIG. 5 is closed. According to FIG. 8, the bottom of the second well 22 is sealed for the second well 22 by protrusions of the second well 22 while both ends of the bottom 30 are sealed with respect to sidewalls, so that liquid of the second well 22 may not move to the first chamber 28.

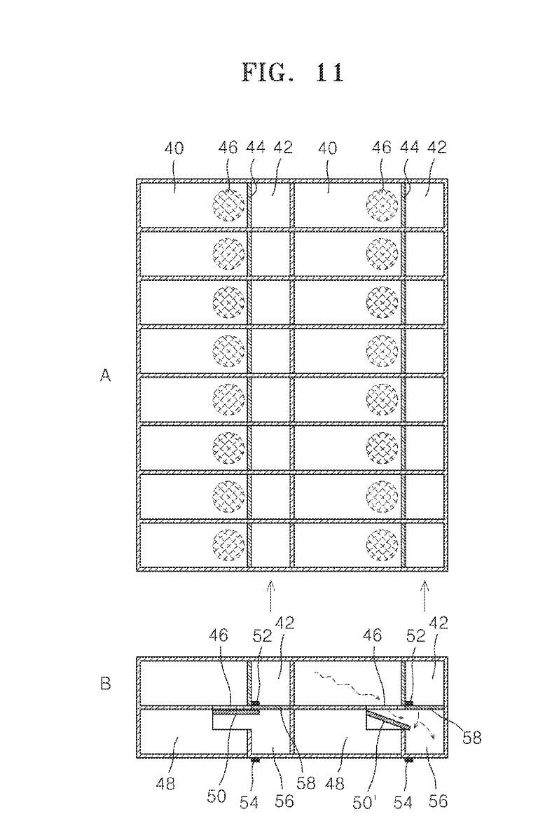

FIG. 9 is a schematic view illustrating a state where the bottom 30 of the second well 22 in the multiwell plate of FIG. 8 is opened. According to FIG. 9, the second well 22 is opened by the bottom 30 thereof rotating downward with the left end of the bottom 30 serving a rotation axis, so that liquid in the first well 20 may be transferred to the first chamber 28 through the pores of the first sidewall 24.

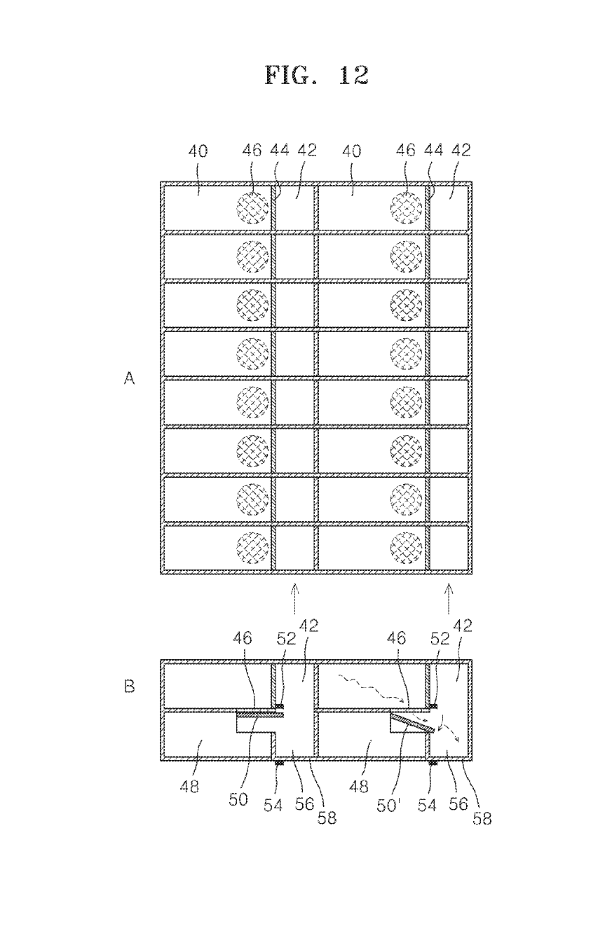

FIG. 10 is a schematic view illustrating a multiwell plate including electrodes (31, 31′) provided at second wells (22, 22′) according to an exemplary example with electrodes prepared for each second well. According to FIG. 10, each of the second wells (22, 22′) may be provided with an electrode (31, 31′) to apply a voltage to each of first sidewalls (24,24′), and the electrodes are connected to a power source and/or controller 32, 34. In case (32) a voltage is not applied, the first sidewall may be closed. Also, in case (34) a voltage is applied, the first sidewall may be opened.

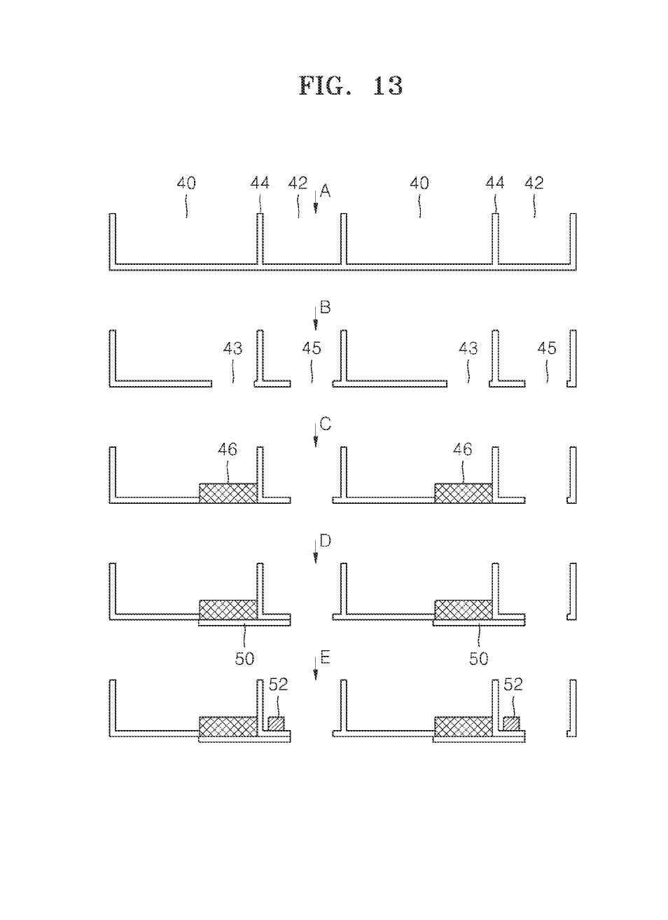

FIG. 11 is a schematic view illustrating a multiwell plate according to another aspect. In FIG. 11, A is a plan view, and B is a sectional view viewed in the arrow direction. As illustrated in FIG. 11, the multiwell plate includes a plate having a top, a bottom, a thickness between the top and the bottom, and a plurality of first wells 40 and second wells 42 extending in the direction of the thickness of the plate, the first wells 40 and at least one of the second wells 42 being separated by a common first sidewall 44, and at least a portion 46 of the bottom of the first well 40 being made of a porous material having one or more pores permeable to liquid component and impermeable to cells, thereby enabling the liquid component to be transferred from the first wells to the outside of the first wells. As illustrated in FIG. 11B, the multiwell plate may include a bottom opening/closing door 50 which is closingly disposed to retain a liquid component in the first well when closed, and to allow the liquid component to leave the first well t through the pores 46 when spaced apart (open). The left side of FIG. 11B shows that the bottom opening/closing door 50 is closingly disposed, and the right side shows that the bottom opening/closing door 50′ is spaced apart (open) with one end thereof serving as a rotation axis. The bottom opening/closing door 50 may be made of a solid supporter or a compressible or elatic material. For example, the bottom opening/closing door 50 may be a solid support that may cover the porous material and be sealable such that liquid may not move downward from and out of the first well. The bottom opening/closing door 50 may be opened or closed mechanically, electrically, or mechanically and electrically. The bottom opening/closing doors 50 may be connected to respective power sources to individually control sealing or spacing thereof. An electrical switch may be connected to each of the bottom opening/closing doors in order to individually control opening or closing of the bottom opening/closing doors. The electrical switch may be a transistor.

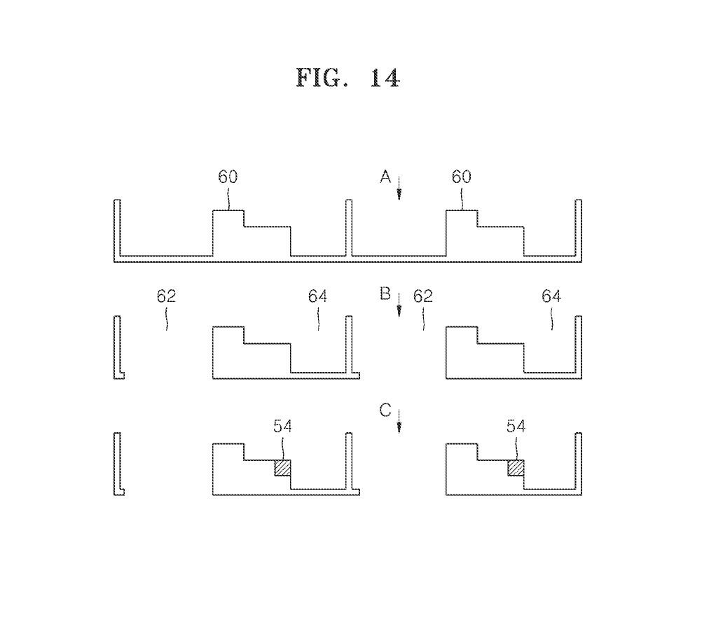

The multiwell plate may have a space (enclosed space or chamber) under the bottom opening/closing door 50 such that the bottom opening/closing door 50 is rotatable and can swing away from the bottom of the first well to open into to the enclosed space. The space may be in fluid-communication with the second well (see FIG. 12B), or may be in fluid-communication with a space or chamber under the second well.

At least a portion under the bottom of the first well 40 may be supported by a supporter 48. The supporter 48 may be a solid supporter that may mechanically support the first well. The solid supporter 48 may be transparent. The solid supporter 48 may be an insulator. Also, the support may be in a vacant space form at a portion other than the bottom opening/closing door under the bottom of the first well in order to facilitate observation of cells growing on the bottom other than the bottom opening/closing door.

The multiwell plate may include two or more electrodes 52, 54, and at least two electrodes may be disposed such that the bottom opening/closing door 50 is located therebetween. One of the two or more electrodes may be disposed on the first sidewall in the second well. At least one electrode may be disposed on the first sidewall 44 in the second well 42, and at least one electrode 54 may be disposed under the bottom of the space 56 (opposite the space or chamber from the second well).

The bottom opening/closing door 50, 50′ may be extended in the second well direction to exceed the extension line of the first sidewall. In other words, when in the closed position, the bottom door is positioned adjacent a bottom surface of the bottom of the first well and extends parallel to the bottom surface in the direction of the second well beyond the extension line of the first sidewall (beyond the point at which the first sidewall joins the bottom of the first well). In this position, the bottom door may extend into the second well (see FIG. 12B), or into a chamber below the second well (see FIG. 11B).

The bottom 46 of the first well may be positioned at the same level as the bottom of the second well. Also, the bottom 58 of the second well may be positioned at a lower level than the bottom 46 of the first well such that the first well is in fluid-communication with the space 56 provided under the bottom opening/closing door 50, 50′.

FIG. 12 is a schematic view illustrating a multiwell plate in which the bottom 58 of the second well is positioned at a lower level than the bottom 46 of the first well according to another aspect. In FIG. 12, “A” is a plan view, and “B” is a sectional view viewed in the arrow direction.

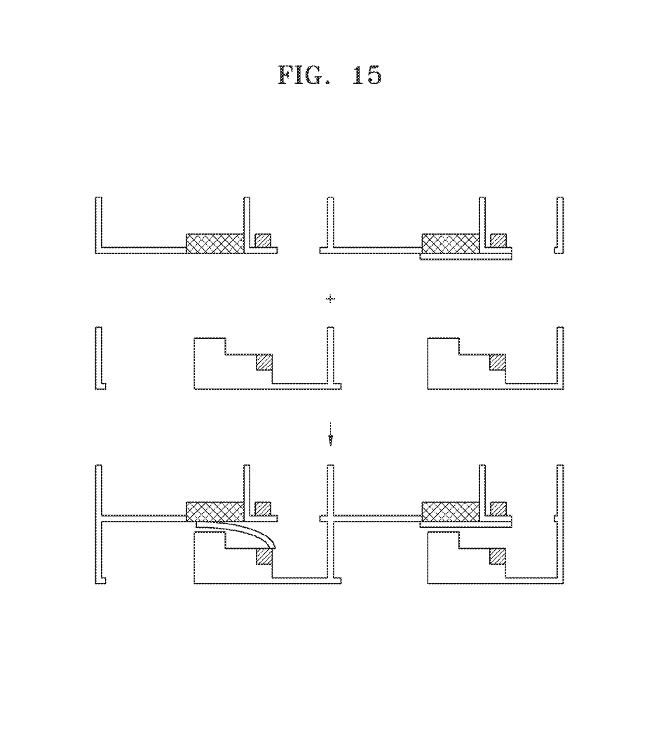

FIGS. 13 to 15 are schematic views exemplarily illustrating a method of manufacturing a multiwell plate according to an embodiment. In detail, FIGS. 13 and 14 are schematic views exemplarily illustrating a method of manufacturing a top plate and a bottom plate of a multiwell plate according to an embodiment. FIG. 15 is a schematic view exemplarily illustrating a method of manufacturing a multiwell plate by joining the top plate and the bottom plate according to an embodiment.

Referring to FIG. 13, first wells 40 and second wells 42 are first formed via molding on a top plate (A). Pores 43, 45 are formed in at least a portion of a bottom of the first wells 40 and at least a portion of a bottom of the second wells 42 to prepare a space for disposing a porous material 46 and a space for suctioning liquid (B). Next, the porous material 46 is attached to the pore (C), and an electroactive material, which forms a bottom opening/closing door 50, is attached under the bottom of the porous material 46. At this time, the bottom opening/closing door 50 may be attached such that it may be disposed under the bottoms of both the first wells and the second wells. Next, an electrode 52 is disposed at or on the bottom of the second wells 42 corresponding to the bottom opening/closing door 50 (E). For a better understanding of FIG. 13, the porous material 46 that it is extruded upwards more than other portions of the bottom of the first wells is exaggeratedly drawn, and the porous material 46 may be smoothly continuously disposed after other portions of the bottom of the first wells.

Referring to FIG. 14, a support structure 60 capable of supporting the bottom opening/closing door 50 is formed on the bottom plate by using a molding (A). A portion of the bottom in the first wells other than the bottom opening/closing door 50 may be removed to enhance light transmittance for observing cell images (B). Also, corresponding portions under the second wells may be removed to prepare spaces for liquid to be stored. Next, an electrode 54 used for opening and closing the door opening/closing door 50 is attached (C). In this embodiment, although the corresponding portions under the bottom of the first wells other than the bottom opening/closing door 50 are removed, the corresponding portions may not be removed if the bottom plate has a sufficient light transmittance.

Next, a multiwell plate is completed by joining the top plate and the bottom plate (see FIG. 15).

As described above, according to the one or more of the above embodiments of the present invention, the multiwell plate may be used for efficient cell culture or analysis.

The cell culture system may be used for efficient cell culture or analysis.

The cell culture system may culture cells efficiently by efficiently exchanging medium.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A multiwell plate comprising a plurality of first wells and a plurality of second wells, each first well being separated from a second well by a common first sidewall,
wherein at least a portion of the first sidewall comprises one or more pores impermeable to cells and permeable to a liquid component, thereby enabling a liquid component to be transferred from the first wells to the second wells.

2. The multiwell plate of claim 1, comprising a first sidewall door on the first sidewall, wherein the first sidewall door is closingly and sealably disposed relative to the one or more pores in the first sidewall, such that the first sidewall door does not allow a liquid component in the first well to move to the second well through the pores when the first sidewall door is closed, and allows a liquid component in the first well to move to the second well through the pores when the first sidewall door is open.

3. The multiwell plate of claim 2, wherein the first sidewall door has a bottom edge nearest the bottom of the second well when closed and a top edge opposite the bottom edge, and the first sidewall door is disposed with a rotational axis along the top edge, such that the bottom edge of the first sidewall door swings away from the first sidewall and into the second well when opened.

4. A cell culture method, comprising:
culturing cells in a medium in a first well of a multiwell plate of claim 1;
transferring the medium in the culture from the first well to a second well through the one or more pores of the first sidewall.

5. The method of claim 4, further comprising removing the medium from the second well, wherein the medium of the second well is removed by opening a bottom of the second well.

6. A multiwell plate comprising a plurality of first wells and a plurality of second wells, each first well being separated from a second well by a common first sidewall,
wherein each first well has a bottom, and at least a portion of the bottom of the first well comprises one or more pores impermeable to cells and permeable to a liquid component, thereby enabling a liquid component to be transferred out of the first well.

7. The multiwell plate of claim 6, wherein the one or more pores is provided by a porous material that has a pore diameter from about 1 μm to about 25 μm.

8. The multiwell plate of claim 6, further comprising a bottom door closingly and sealably disposed relative to the one or more pores in the bottom of the first well, such that the bottom door does not allow a liquid component in the first well to move out of the first well through the pores when closed, and allows a liquid component in the first well to move out of the first well through the pores when open.

9. The multiwell plate of claim 8, wherein the bottom door is disposed with a rotational axis along one edge, such that the door swings away from the bottom of the first well in a direction away from the first well.

10. The multiwell plate of claim 9, wherein the bottom door is opened or closed mechanically, electrically, or electromechanically.

11. The multiwell plate of claim 8, wherein the bottom door comprises an electroactive material that bends when a voltage is applied thereto, such that the bottom door opens or closes upon application of a voltage.

12. The multiwell plate of claim 6, further comprising an enclosed space separated from the first well by the bottom of the first well, wherein the bottom door opens into the enclosed space.

13. The multiwell plate of claim 8, comprising at least two or more electrodes,
wherein the at least two or more electrodes are disposed such that the bottom door is disposed between the at least two or more electrodes to allow to apply a voltage to the bottom door.

14. The multiwell plate of claim 8, wherein the bottom door, when closed, is positioned adjacent a bottom surface of the bottom of the first well and extends parallel to the bottom surface in the direction of the second well beyond an extension line of the first sidewall.

15. The multiwell plate of claim 12, wherein the bottom of the second well is lower than the bottom of the first well and is in fluid communication with the enclosed space into which the bottom door opens.

16. The multiwell plate of claim 8, wherein the closing or opening of the bottom door is individually controllable.

17. A cell culture method, comprising:
culturing cells in a medium in a first well of a multiwell plate of claim 6 and transferring the medium in the culture from the first well through the one or more pores of the bottom of the first well.

18. The method of claim 18, wherein the multiwell plate is a multiwall plate of claim 8, and the medium is transferred by opening the bottom door.

19. The method of claim 19, wherein the bottom door is opened mechanically, electrically, or electromechanically.

20. The multiwall plate of claim 1 further comprising a chamber below the second well and separated from the second well by a bottom of the second well perpendicular to the first sidewall, wherein the bottom of the second well is closingly disposed relative to the chamber.
